(19) United States
(12) Patent Application Publication
(10) Pub. No.: US 2022/0265566 A1
(43) Pub. Date: Aug. 25, 2022

(54) ATOVAQUONE NANOPARTICULATE COMPOSITIONS

(71) Applicant: Tulex Pharmaceuticals Inc., Cranbury, NJ (US)

(72) Inventors: Yu-Hsing Tu, West Windsor, NJ (US); Yingjun Fan, Plainsboro, NJ (US); Kalyan Kathala, Monroe, NJ (US); Ashok Perumal, Monmouth Junction, NJ (US)

(21) Appl. No.: 17/286,349

(22) PCT Filed: Oct. 18, 2019
(86) PCT No.: PCT/US2019/057030

§ 371 Date: Apr. 16, 2021

Related U.S. Application Data
(60) Provisional application No. 62/747,504, filed on Oct. 18, 2018.

(51) Int. Cl. A61K 9/51; A61K 31/122; A61K 31/155; A61K 31/7052; A61K 31/435

(52) U.S. Cl. CPC A61K 9/5146; A61K 31/122; A61K 31/155; A61K 31/7052; A61K 31/435; A61K 9/0053

(57) ABSTRACT

A nanoparticle ATQ composition is provided which has good stability and bioavailability. Compositions and methods of using the nanoparticle ATQ composition in treating parasitic and other infections is described.

### BACKGROUND OF THE INVENTION

Atovaquone (ATQ) (566C80) is a quinone antimicrobial drug, having the chemical name trans-2-[4-(4-chlorophenyl) cyclohexyl]-3-hydroxy-1,4-naphthalenedione. The structure of this compound is as follows:

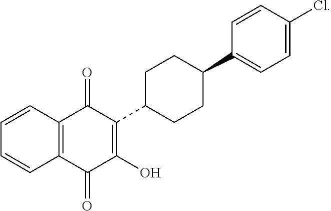

See, MEPRON® (ATQ oral suspension) product literature. The structure of this and other 2-substituted-3-hydroxy-1,4-napthoquinones described in U.S. Pat. No. 5,053,432. ATQ was first released in 1992 as a tablet, but was discontinued because of poor and unreliable bioavailability [A. L. Baggish and D R Hill, Minireview Antiparasitic Agent ATQ, Antimicrobial Agents and Chemotherapy, Vol 46, No. 5, May 2002, p. 1163-1173]. The drug was then reformulated as a micronized suspension and was marketed as MEPRON® ATQ oral suspension. Baggish and Hill, cited above. ATQ is an agent with antiparasitic characteristics for the treatment of pneumocystis pneumonia [Hughes W, et al, Comparison of ATQ with trimethoprim-sulfamethoxazole to treat Pneumocystis carinii pneumonia in patients with AIDS. N. Engl. J. Med. 1993;328:1521-7; Hughes W T, et al, Effects of aerosolized synthetic surfactant, ATQ, and the combination of these on murine Pneumocystis carinii pneumonia. J. Infect. Dis. 1998;177:1046-56], malaria [Mulenga M, et al, Atovaquone and proguanil versus pyrimethamine/sulfadoxine for the treatment of acute falciparum malaria in Zambia. Clin. Ther. 1999;21:841-52] and toxoplasmosis Djurković-Djaković O, et al, Efficacy of atovaquone combined with clindamycin against murine infection with a cystogenic (Me49) strain of Toxoplasma gondii. J. Antimicrob. Chemother. 2002;50:981-7]. It is a structural analogue of protozoan ubiquinone (also known as coenzyme Q) that targets the mitochondrion by inhibiting the electron transport and causes the collapse of the membrane potential in such organelles [Baggish A L, Hill D R. Antiparasitic agent atovaquone. Antimicrob. Agents Chemother. 2002; 46:1163-73]. ATQ is practically insoluble in water and belongs to class II in the bio pharmaceutics classification system (BCS) which exhibits a low aqueous solubility due to the hydrophobic nature but high membrane permeability. Dissolution of the drug in the intestinal medium is the rate-limiting step of absorption [Fleisher D, et al., Drug, meal and formulation interactions influencing drug absorption after oral administration. Clin. Pharmcokinet. 1999;36:233-54].

ATQ has been previously incorporated into several types of nano formulations including nanocapsules, nanosuspension and liposomes [Cauchetier E, et al, Atovaquone-loaded nanocapsules: influence of the nature of the polymer on their in-vitro characteristics. Int. J. Pharm. 2003; 250:273-81; Schöler N, Krause et al, Antimicrob. Agents Chemother. 2001;45:1771; Cauchetier E, et al, Preparation and physicochemical characterization of atovaquone-containing liposomes. Drug Dev. Res. 1999; 47:155-61]. N. Mohtar et al, Iran J Pharm Res, 2015 Autumn; 14(4): 989-1000 reports that a solid lipid nanoparticle formulation offers several advantages including the ability to increase lipophilic drug bioavailability and controlled-release delivery and describes solid lipid nanoparticles based on 2⁴ full-factorial design. The formulation can be a solution to avoid bio toxicity problems as it uses physiological lipids such as triglycerides and natural surfactants such as soy lecithin which are generally recognized as safe (GRAS).

Nanosuspensions have been described as biphasic, colloidal dispersions of drug particles which are stabilized by using surfactants and which can overcome the problems related to delivery of poorly water-soluble drugs. See, M. Sharma, et al, “Nanotechnology Based Approaches for Enhancing Oral Bioavailability of Poorly Water Soluble Antihypertensive Drugs”, Scientifica, Vol. 2016, pp. 1-12. Various methods for producing drug nanoparticle suspensions has been described. See, e.g., A. Monteiro, et al, Drug Development and Industrial Pharmacy, 2013; 39(2): 266-283 which describes continuous production of drug nanoparticle suspension via wet stirred media milling to produce suspension with particles having a mean or medium size in the range of 1 to 10 μm. T. Niwa, et al, Pharm Res (2011): 28: 2339-2349 describes powderizing the aqueous nanosuspension of a poorly-water soluble drug, which was prepared by wet-milling techniques. See, also, Z H Loh, et al, “Overview of milling techniques for improving the solubility of poorly water-soluble drugs”, Asian J Pharm Sci, 10 (2015): 255-274. Processing equipment includes, e.g., planetary ball mill, vibratory media mill, wet stirred media mill, high energy media mill. See, M. Li et al, Pharmaceutics, 2016, Vol 8 (17), p. 1-35.

A commercially available ATQ suspension is available as MEPRON oral suspension in doses of 10 mg/kg, 30 mg/kg, and 45 mg/kg. According to the product literature, MEPRON oral suspension is a formulation of micro-fine particles of ATQ and each 5 mL of MEPRON oral suspension contains 750 mg of ATQ and the inactive ingredients benzyl alcohol, flavor, poloxamer 188, purified water, saccharin sodium, and xanthan gum. The FDA Orange Book listed U.S. Pat. No. 6,649,659 as covering this product. The '659 patent claims small particles of ATQ wherein at least 90% of the particles of ATQ have a volume diameter in the range 0.1-3 μm. The specification describes preparing microfluidized particles of ATQ, which are composed of a mixture of ATQ in aqueous Celacol M2500, a methylcellulose emulsifying and suspending agent. See, U.S. Pat. No. 6,018,080 for methods of preparing microfluidized particles of ATQ with a liquid vehicle. According to the '080 patent, at least 90% of the particles have a volume diameter in the range of 0.1 μm to 3 μm, and preferably at least 95% of the particles have a volume diameter in the range 0.1 to 2 μm.

The commercial product has been described as being made by microfluidizer technology, which involves the use of high pressure homogenizers converting extreme fluid pressures into shear forces to reduce the particles suspended in liquid media. However, the literature has described several disadvantages of such microfluidizer. See, e.g., page 79 of “Nanoparticulate Drug Delivery Systems” edited by Deepak Thassu, Michael Deleers, Yashwant Pathak, published by CRC Press, 2007 Taylor & Francis Group, which reports that the long production time required for microfluidizers is a major disadvantage. See, also, R. Mohammadi, etc. “Applications of nanoliposomes in cheese technology”, International Journal of Dairy Technology, vol 68, No. 1 February 2015, Page 11-23; S. Silvestri, et al., “Effect of terminal block on the microfluidization induced degradation of a model A-B-A block polymer”, International Journal of Pharmaceutics, 71(1991) 6-71; reports that a major disadvantage of microfluidization is the high number of passes through the microfluidizer and that the product obtained contains a relatively larger fraction of microparticles. See, also, “Lipid Nanoparticles: Production, Characterization and Stability” by Rohan Shah, Daniel Eldridge, Enzo Palombo, Ian Harding, Springer International Publishing.

What are needed are ATQ products which have improved stability, purity, and/or enhanced bioavailability.

### SUMMARY OF THE INVENTION

Advantageously, the ATQ compositions provided herein have improved bioavailability.

In one aspect, a nanoparticle atovaquone composition is provided which comprises: (a) an atovaquone dispersion comprising atovaquone nanoparticles and at least one nanoparticulate pharmaceutically acceptable dispersant based on the total atovaquone dispersion, having a particle distribution of: wherein at least about 90% of the atovaquone nanoparticles have a volume diameter below about 0.776 μm, as determined using dynamic light scattering (DLS), at least about 50% of the nanoparticles have a volume diameter below about 0.549 μm; at least about 10% of the nanoparticles have a volume diameter below about 0.393 μm; and (b) a gum dispersion. In certain embodiments, the dispersion contains as the dispersant is poloxamer 188, poloxamer 238, poloxamer 407, or a mixture thereof. Optionally, the dispersant component comprises a viscosity enhancing component and/or an ionic species, optionally further in combination with a surfactant.

In a further embodiment, a nanoparticle powder suitable for preparing a suspension is provided, which comprises: (a) an atovaquone dispersion comprising atovaquone nanoparticles and poloxamer 188 nanoparticles based on the total atovaquone dispersion, having a particle distribution of: wherein at least about 90% of the atovaquone nanoparticles have a volume diameter below about 0.776 μm, as determined using dynamic light scattering (DLS), at least about 50% of the nanoparticles have a volume diameter below about 0.549 μm; at least about 10% of the nanoparticles have a volume diameter below about 0.393 μm.

In certain embodiments, a pharmaceutically acceptable composition comprising a combination of nanoparticle atovaquone and proguanil is provided. This composition may be a solid or a liquid suspension which comprises: (a) an atovaquone dispersion comprising atovaquone nanoparticles and at least one nanoparticulate pharmaceutically acceptable surfactant, wherein based on the total atovaquone dispersion, having a particle distribution of: wherein at least about 90% of the atovaquone nanoparticles have a volume diameter below about 0.776 μm, as determined using dynamic light scattering (DLS), at least about 50% of the nanoparticles have a volume diameter below about 0.549 μm; and at least about 10% of the nanoparticles have a volume diameter below about 0.393 μm, and proguanil.

In a further aspect, a method for treating malaria in a human is provided. The method comprises administering a therapeutically effective amount of a composition comprising nanoparticulate atovaquone as described herein.

In yet another aspect, a method for treatment of a Babesia, HIV infection, and/or AIDS is provided. This method comprises co-administering a nanoparticulate atovaquone composition as provided herein to a patient in need thereof In certain embodiments, azithromycin is co-administered with the atovaquone composition.

In a further aspect, a method for treatment of parasitic and/or tick-borne infections of companion animals is provided. This method comprises administering a nanoparticulate composition comprising atovaquone as provided herein to an animal in need thereof. In certain embodiments, azithromycin is co-administered with the atovaquone composition.

In yet a further aspect, a method for treating Pneumocystic carinii infections is provided. This method comprises co-administering a composition comprising atovaquone as provided herein and rifabutin. In certain embodiments, the rifabutin is in the atovaquone suspension. In other embodiments, the rifabutin is formulated separately from the atovaquone suspension.

These and other advantages of the invention will be apparent from the following detailed description of the invention.

### BRIEF DESCRIPTION OF THE DRAWINGS

FIG. 1 is a line graph showing the results of a biostudy comparing two test ATQ nanosupension formulations in comparison to a reference product, a commercially approved microfluidized MEPRON suspension. The mean plasma concentration of ATQ is shown over the test period (240 hr).

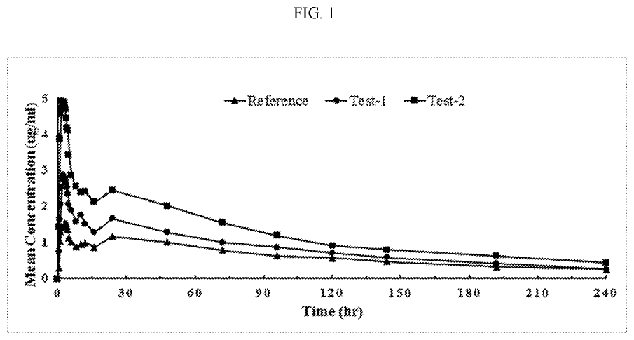

### DETAILED DESCRIPTION OF THE INVENTION

Described herein are compositions containing pharmaceutically useful quinine and/or atovaquone nanoparticles e.g., ATQ) which have improved bioavailability, which are useful for treating a variety of indications, including, without limitation, malaria, pneumocystic pneumonia (PCP), toxoplasmosis, and Babesia.

As used herein, “atovaquone” refers to 2-substituted-3-hydroxy-1,4-naphthoquinones of formula:

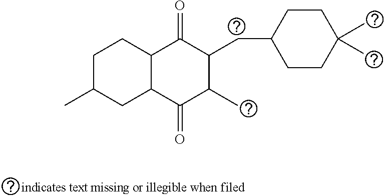

wherein either R¹ is hydrogen and R² is selected from C₁₋₆ alkoxy, aralkoxy, C₁₋₆ alkyl-C alkoxy, phenyl substituted by one or two groups selected from halogen and C₁₋₆ alkyl, halogen and perhalo-C₁₋₆ alkyl or R¹ and R² are both C₁₋₆ alkyl or phenyl, and n is zero or 1, and any physiologically acceptable salts thereof. Atovaquone and its pharmaceutically acceptable salts are abbreviated “ATQ” throughout the specification, unless otherwise specified. In certain embodiments, atovaquone has the chemical name: trans-2-[4-(4-chlorophenyl)cyclohexyl]-3-hydroxy-1,4-naphthalenedione has the structure:

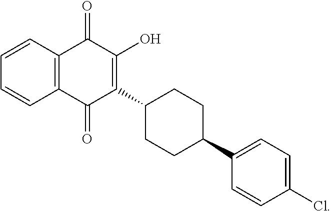 [SMILES: O=C1C(O)=C([C@H]2CC[C@H](c3ccc(Cl)cc3)CC2)C(=O)c2ccccc21]

In certain embodiments, an enantiomers of this compound or racemic mixture containing this compound may be selected.

As used herein, an “ATQ dispersion” is a nanodispersion comprising the ATQ free base or salt, admixed with at least one excipient, which is milled to a size in which the average total surface area of the particles is at least 5500 m²/kg, e.g., about 5500 m²/kg to about 12,000 m²/kg, or at least about 10,000 m²/kg. In certain embodiments, an ATQ nanodispersion is provided in which at least about 90% of the ATQ nanoparticles have a mean volume diameter below about 0.776 μm, as determined using dynamic light scattering (DLS); of which at least about 50% of the nanoparticles have a mean volume diameter below about 0.549 μm and at least about 10% of the nanoparticles have a mean volume diameter below about 0.393 μm. In certain embodiments, an ATQ nanodispersion is provided in which at least about 90% of the ATQ nanoparticles have a mean volume diameter below about 5.61 μm, as determined using dynamic light scattering (DLS); of which at least about 50% of the nanoparticles have a mean volume diameter below about 1.81 μm and at least about 10% of the nanoparticles have a mean volume diameter below about 0.4233 μm, wherein the total ATQ nanodispersion has a total surface area of at least 5250 μm. In certain embodiments, these values are determined using a commercially available particle size analyzer, such as measured by Malvern Instrument Model 3000, Mastersizer 3000 using standard settings or another suitable device.

Thus, in certain embodiments, an ATQ nanodispersion includes the ATQ dispersion having the subpopulations of the particle sizes ranges for ATQ provided herein and a dispersant. As used herein, a “dispersant” or “dispersing agent” is either a surface active or non-surface-active agent which improves the separation of the particles and prevent settling or clumping, e.g., of the ATQ. Suitable pharmaceutically acceptable dispersants are non-ionic or ionic and exhibit acceptable stability in the presence of acids, alkalis and metal ions. Further, dispersants provide acceptable steric stabilization for ATQ (and any optional further drug) during the milling process. Suitable non-surface active agents may include ionic species and/or viscosity enhancing materials which prevent agglomeration.

In certain embodiments, a dispersant is selected which aids in preventing agglomeration (aggregation or flocculation) of ATQ during the milling process and/or dispersion stage. Agglomeration may be measured by particle size changes using dynamic light scatter techniques and/or visual observation. See, e.g., J M Zook, et al, Nanotoxicolory, December 2001; 5(4): 517-530.

In certain embodiments, a dispersant has a zeta potential which is at about ±20 mV to about ±60 mV or more, about ±20 mV to ±60 mV or more, when assessed at a concentration of about 0.5% w/v to 2% w/v. In certain embodiments, the dispersant(s) have a zeta potential of about ±30 mV to about ±55 mV, ±30 mV to ±55 mV, about ±40 mV to about ±50 mV, or ±40 mV to 50 mV, when assessed at a concentration of 0.5 w/v % to 2% w/v.

Examples of suitable dispersants include, without limitation, surfactants such as poloxamers. In certain embodiments, the dispersant selected is a surfactant such as a poloxamer. Particularly suitable poloxamers include, poloxamers 188, 237, 238, or 407. Poloxamer is a synthetic block copolymer of ethylene oxide and propylene oxide having the following structure:

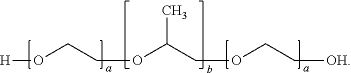

For poloxamer 188, a is 12 and b is 20 in the preceding structure. Poloxamer 188 is solid at room temperature, has an average molecular weight of 7680 to 9510, a weight percent of oxyethylene of 81.8±1.9, and an unsaturation of 0.026±mEq/g. P188 is also known commercially as Kolliphor®188, formerly known as Lutrol® F 68, Pluronic® F-68, Flocor™ and has an average molecular weight of about 8400 g/mol. For poloxamer 237, a is 64 and b is 37. Poloxamer 237 is a solid at room temperature and has an average molecular weight of 6840 to 8830, a weight percent of oxyethylene of 72.4±1.9, and an unsaturation of 0.034±0.008. For poloxamer 338, a is 141, b is 44; is a solid at room temperature, has an average molecular weight is 12700 to 17400, weight percentage of oxyethylene is 83.1±1.7, unsaturation is 0.031±0.008 mEq/g. For poloxamer 407, a is 101 and b is 56; solid at room temperature, average molecular weight of 9840 to 14600, weight percentage oxyethylene of 73.2±1.7, unsaturation of 0.048±0.017 mEq/g. See, USP29, U.S. Pharmacopeia. A single poloxamer may be selected, a combination of poloxamers may be selected, or one or more poloxamers may be combined with another surfactant.

| | Concentration | |
|---|---|---|
| Surfactant | (W/V %) | Zeta Potential (mV) |
| Kolliphor ® P188 [BASF brand]: CAS-No: | 2 | −19.4 ± 0.6 |
| 9003-11-6 | | |
| Former trade name: Lutrol ® F 68 | | |
| Kollisolv ® P124[BASF brand] polyethylene- | 2 | −39.1 ± 0.4 |
| polypropylene glycol containing d,l-alpha | | |
| tocopherol as an antioxidant.-CAS# 9003-11-6 | | |
| Kolliphor ® P338 [BASF brand] (formerly | 2 | −31.7 ± 1.0 |
| Lutrol F 108) methyl oxirane polymer with | | |
| oxirate, with antioxidant BHT | | |
| Kolliphor ® P407 [BASF brand] formerly | 2 | −39.0 ± 0.4 |
| Lutrol F 127, Polyoxyethylene (196) | | |
| Polyoxylpropylene (67) glycol, CAS-No. | | |
| 9003-11-6 | | |
| Kolliphor ® PS20 [BASF brand] CAS-No: | 0.53 | −54.5 ± 0.6 |
| 9003-64-5, polysorbate 20, | | |
| Kolliphor ® PS60[BASF brand] | 0.53 | −46.2 ± 0.3 |
| Polyethylene(20)sorbitanmonostearate-CAS# | | |
| 9005-67-8 | | |
| Kolliphor ® PS80 [BASF brand] polysorbate | 0.53 | −50.4 ± 0.3 |
| 80, CAS-No: 9005-65-6 | | |

Still other surfactants or other moieties may be selected for use as dispersants. For example, suitable surfactants may include, e.g., sodium dodecyl sulphate (SDS), poly(ethylene oxide)20 sorbitan monolaurate (Tween 20), sorbitan monooleate (Tween 80). Examples of pharmaceutically acceptable surfactants may include those described in, e.g., B S Shekon, J Pharm Tech, Research and Management (JPTRM), Vol 1, May 2013, pp 11-36. In certain embodiments, other excipients may be blended with the dispersant. Optionally, the milling process may utilize a processing aid, e.g., benzyl alcohol.

Examples of ionic species which may be used alone, or in combination with another component, as a dispersant include commonly used anions and cations typically used in formation of non-toxic salts. Suitable ionic species are formed from acids which form non-toxic salts. Examples include the acetate, lactobionate, benzenesulfonate, laurate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandalate, bitartrate, methylbromide, bromide, methylnitrate, calcium edetate, mucate, napsylate, chloride, clavulanate, Butyl(N) oleate, edetate, estolate, pantothenate, polygalacuronate, salicylate, glutamate, glycollylarsanilate, sulfate, hexylrosorcinate, subacetate, hydrabamine, hydroxynaphthaloate, etolate, triethiodide, valerate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable ionic species are formed from bases which form non-toxic salts. Examples include the aluminum, arginine, benzathine, calcium, choline, diethylamine, diolamine, glycine, lysine, magnesium, meglumine, olamine, ornithine, N,N-dibenzyethelenediamine, piperazine, tris(hydroxymethyl)aminomethane, tetramethylammonium hydroxide, methylglucamine, ammonium salt, potassium, sodium, tromethamine, 2-(diethylamino)ethanol, ethanolamine, morpholine, 4-(2-hydroxyethyl)-morpholine and zinc salts. Particularly suitable ionic species include hydrochloride/chloride, hydrobromide/bromide, bisulphate/sulphate, nitrate, citrate, and acetate.

Suitable viscosity enhancing materials for use as a dispersant, either alone or in combination with another dispersant, include pharmaceutical ingredients which function as thickeners, texturizer, gelation agents and stiffening agents. Examples of suitable products include, e.g., Compritol 888 ATO, Monosteal, Precirol ATO, maltodextrin, glycolate and derivates thereof (e.g., sodium starch glycolate), natural gums (e.g. acacia, tragacanth, alginic acid, sodium alginate, carrageenan, arabic gum, locust bean gum, dammar gum, karaya, guar gum, gellan gum, xantham gum, agar, etc), cellulose derivatives (e.g., carboxymethylcellulose, methylcellulose, ethyl cellulose, hydroxy ethyl cellulose, hydroxypropyl methyl cellulose, etc) microcrystalline cellulose, chitosan, synthetic polymers (e.g., carbomer (polyacrylic acid), polyvinylpyrrolidone, polyvinylalcohol, etc), and clays (magnesium aluminum silicate (veegum), bentonite, attapulgite).

In certain embodiments, during the milling process, the about 5% w/w total dispersant(s) to about 20% w/w total dispersant(s), based on the solids content of the final dispersion may be present. During the milling process, together with the ATQ (and any optional additional drug), there may optionally be one or more excipients and some liquid content which may or may not be removed for processing the dispersant into the final formulation. In certain embodiments, more than one dispersant is provided. In certain embodiments, more than one type of dispersant (e.g., an ionic species, viscosity enhancing material, or a surfactant) is provided. In certain embodiments, two or more dispersants from at least one type of dispersant are combined with at least one dispersant from at least one other type (e.g., two or more surfactants with a gum and a surfactant; or two or more viscosity enhancers and a surfactant; an ionic species and two or more gums; or two or more surfactants and a viscosity enhancer).

In certain embodiments, ATQ nanosuspensions are provided which have a Cₘₐₓ of about 2.5 μg/mL to about 10.8 μg/mL for the ATQ. In certain embodiments, ATQ nanosuspensions are provided which have an AUCᵢ of about 113.5 μg·hr/mL to 686.6 μg·hr/mL for the ATQ.

In certain embodiments, compositions containing an additional or different pharmaceutically useful compound such as buparvaquone may be prepared as described herein. The chemical structure of buparvaquone is as follows:

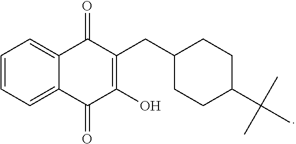 [SMILES: CC(C)(C)C1CCC(CC2=C(O)C(=O)c3ccccc3C2=O)CC1]

In certain compositions containing an additional or different pharmaceutically useful compound such as parvaquone may be prepared as described herein. The chemical structure of parvaquone is as follows:

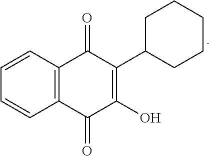 [SMILES: O=C1C(O)=C(C2CCCCC2)C(=O)c2ccccc21]

As used herein, the term “bioavailability”, is the degree and rate at which a substance (such as a drug) is absorbed into a living system or is made available at the site of physiological activity. In certain embodiments, relative bioavailability is determined by comparing one or more pharmacokinetic parameters of a test product to one or more pharmacokinetic parameters of a reference product. For example, one suitable measure of relative bioavailability may be AUC_{inf}.

For example, in certain embodiments, ATQ nanoparticle compositions are provided which have at least about 30% higher bioavailability as compared to the ATQ MEPRON® oral suspension product. In certain embodiments, ATQ nanoparticle compositions are provided which have at least about 2-fold higher (double or 100% higher) bioavailability as compared to the ATQ MEPRON® oral suspension product. See, for example, the Test/Reference Ratios provided in the Table of Example 4. Thus, in certain embodiments, the dose of an ATQ composition as provided herein may provide a lower dose of ATQ than the commercially available product to achieve the same therapeutic effect. In certain embodiments, the dose may be about 30% to 250% lower doses of ATQ. However, other doses may be selected as needed or desired.

In certain embodiments, the AUC values are calculated by the linear trapezoidal rule.

“Cₘₐₓ” is the maximum plasma concentration, calculated as the geometric or arithmetic mean of the individual maximum blood plasma concentrations over the sampling period.

The term “AUCᵢ” or “AUC_{inf}” or “AUG_{0-∞}” is the mean area under the analyte (e.g., from plasma or serum) concentration-time curve extrapolated to infinity It is calculated as the mean of the area under the analyte concentration-time curve from time 0 extrapolated to infinity, calculated for each individual participating in the bioavailability study and may be the geometric or arithmetic mean. In certain embodiments, AUC0-∞=AUC0-t+Ct/kel, where Ct is the last measurable analyte concentration and kel is the apparent first-order elimination rate constant.

AUCₜ or AUC₀₋ₜ is the area under the plasma/serum/blood concentration-time curve from time zero to time t, where t is the last time point with measurable analyte concentration.

“Bioequivalent” means the pharmacokinetic profile of a test composition is within the range of about 80% to about 125%, when compared to the values of one or more of the AUC values or the Cₘₐₓ of the reference composition. See, e.g., US Department of Health and Human Services. Guidance for Industry: Bioavailability and Bioequivalence Studies for Orally Administered Drug Products—General Considerations. Washington, DC: Center for Drug Evaluation and Research; March 2003. For example, in certain embodiments, a bioequivalent product is required to be within 80% to 125% of AUC_{0-∞}, or within 90% to 110% of AUC_{0-∞}. In certain embodiments, a bioequivalent product is bioequivalent over preselected partial AUC values. Typically, in order to be bioequivalent, a product must match not just a single partial AUC, but also either be bioequivalent over the entire time period and/or other partial AUC time periods as pre-defined.

In order to declare the absence of a food effect on the test formulation, the 90% CIs of the relative mean plasma concentration of one or more of AUCₜ and/or Cₘₐₓ of the test product under fed versus fasted conditions should be between 80.00 and 125.00%, inclusive. See, e.g., US Department of Health and Human Services. Guidance for Industry: Food Effect Bioavailability and Fed Bioequivalence Studies. Rockville, Md.: Center for Drug Evaluation and Research; December 2002. In one embodiment, a composition of the invention has no food effect, i.e. there is no statistical difference in one or more of the pharmacokinetic parameters, including, e.g., Cmax, AUC0-∞, and/or one or more partial AUCs, when patients are administered a dose under fasted conditions as compared to fed conditions.

In order to establish relative bioavailability of a test formulation (e.g., administered as a whole tablet to the reference or chewing under fasted conditions), the 90% confidence intervals (CIs) of the ratios of geometric mean plasma of the test product in relation to the reference product should be between 80.00 and 125.00%, inclusive. See, e.g., US Department of Health and Human Services. Guidance for Industry: Bioavailability and Bioequivalence Studies for Orally Administered Drug Products—General Considerations. Washington, DC: Center for Drug Evaluation and Research; March 2003.

The term “half-life” is the apparent terminal elimination half-life (T_{1/2}).

The words “comprise”, “comprises”, and “comprising”, and “contain”, “containing”, and “contains” are to be interpreted inclusively rather than exclusively. The words “consist”, “consisting”, and its variants, are to be interpreted exclusively, rather than inclusively.

As used herein in reference to numeric values provided herein, the term “about” may indicate a variability of as much as 10% (up to ±10% of the indicated value, including). This includes lower variabilities (e.g., ±1%, ±2%, ±3%, ±4%, ±5%, ±6%, ±7%, ±8%, ±9%, and other values between 0 and 10).

### Production/Manufacture

In one object, a vertical milling process provides the advantages of tighter particle size distribution, higher drug loading, less process time, less contamination chances (by requiring less media and reducing media contact with the process vessel), particularly useful for the ATQ, and the quinone and quinine drugs identified herein that are unstable in the milling environments such as high pressure, high temperature, long process time, etc. and overall better efficient process. Advantageously, resulting wet milled drug(s) can be further processed to prepare suspension with desirable strengths, or mixed with appropriate excipients to form granules and prepared into solid dosage form such as powder, tablet, capsules, etc.

In certain embodiments, one or more of the drugs described herein can be prepared as nanoparticles through the use of equipment and methods. Examples of nanomilling procedures and equipment have been described in the literature. See, e.g., M. Li et al, Pharmaceutics, 2016: 8, 17: 1-35, Vijaykumar Nekkanti and Javier Rueda (2016) Nanoparticles for Improved Delivery of Poorly Soluble Drugs. J. Drug 1(1): 18-27. doi: https://doi.org/10.24218/jod.2016.4., and Z H Loh, et al, Asian Journal of Pharmaceutical Sciences, Vol 10, Issue 4, July 2015, pp. 255-274, which are incorporated by reference in its entirety. In certain embodiments, a basket media mill, also known as an immersion mill is selected. Such mills have been described in the literature as combining mixing and milling, enabling the grinding and dispersion process to take place in a temperature-controlled milling chamber. See, e.g., U.S. Pat. Nos. 5,184,783 and 8,376,252. Such mills may be obtained from commercial sources, including, e.g., Netzsch Inc, Kady International, Konmix [China], Hochmeyer, Oliver and Batlle, VMA-Getzmann, among others. Basket media mills of the type have also been sold by Asada Iron Works Co., Ltd. of Osaka, Japan. A somewhat similar apparatus is sold by Mirodur S.p.A. of Italy, under the designation “TURBOMILL”. Generally, a media basket mill is useful for dispersing a selected solid material into a liquid vehicle to produce a mixture of the solid and the liquid vehicle within a mixing vessel, the media basket mill comprising: a basket having an interior and a basket wall including an interior surface and an exterior surface, the basket extending axially between a first end and a second end and including an entrance at the second end; a multiplicity of openings in the basket wall, the openings extending from the interior surface to the exterior surface of the basket wall; a media bed in the interior of the basket, the media bed including discrete media elements, the relative dimensions of the media elements and the openings in the basket wall being such that the media bed is retained within the interior of the basket; means for establishing a first pressure differential adjacent the first end of the basket for moving the mixture along a circuit through the basket, and through the media bed in the basket; and means for establishing a second pressure differential adjacent the second end of the basket for assisting in the movement of the mixture along the circuit into the entrance to the basket and through the basket, and through the media bed in the basket, to increase the throughput of the mixture through the basket and the media bed in the basket, while deterring escape of media elements from the basket through the entrance at the second end of the basket. Useful variations of these equipment and methods will be readily understood by one of skill in the art. This method may also be referred to as “wet milling”.

In certain embodiments, an ATQ dispersion is milled using an immersion mill having a chamber with a porous wall and the temperature being maintained at about room temperature (e.g., about 22° C. to about 28° C.). A stir paddle may be used to improve recirculation during the milling process. Typically, the milling process involves the use of “beads” or “grinding media” which is loaded in to the mill with the drug dispersion. In certain embodiments, the grinding media is composed of spherical beads having the desired size and are pharmaceutically acceptable. In certain embodiments, about 20% to about 90% of the capacity of the milling chamber is filled with the grinding media. In other embodiments, the amount is about 30% to about 70%, or about 70%.

Typically, the grinding media selected is non-porous, chemically inert, chip-resistant and hard. Certain media selected is also non-conductive and non-magnetic, such as the zirconium oxide described in some of the examples. Zirconium oxide media is available from a variety of sources including, e.g., Zircoa [see, e.g., physical properties of ceramic grinding and milling at www.zircoa.com/product.fine.grain/-grinding.media.html] or Glen Mills [www.glenmills.com/grinding-media/], including various fused, stabilized (magnesium, rare earth, yttrium), high density or other zirconium silicates. Other types of grinding media may be selected for preparing the nanoparticles described herein. The size of the grinding media may be in the range of 0.2 mm to about 0.3 mm. However, depending upon the nanoparticle size desired for the selected drug, the size of the grinding media may be varied.

The mixture may be recirculated periodically or continuously through the chamber. The milling speed is generally in the range recommended by the manufacturer and may be proceed for about 10 minutes to about 120 minutes. However, shorter or longer times maybe used. Further, in certain embodiments, milling may be at a higher speed for a short period of time, a lower speed for a longer period of time, or combinations of higher and lower speeds may be used in combination with different durations of milling time at each speed. Following completion of milling, the ATQ dispersion may be combined with suitable components to prepare a final formulation.

Advantageously, through the combination of wet milling of the drug(s) and the use of the pharmaceutically acceptable dispersants identified herein, the ATQ nanoparticles provided herein are in the nano-sized range. Suitably, these nanoparticles are also well separated, i.e., not agglomerated or aggregated.

According to the present invention, an ATQ nanodispersion is prepared by wet milling ATQ and a poloxamer solution in a stainless steel jacketed container using an overhead stirrer to form a uniform dispersion. In general, the particles are milled to a size such that the resulting dispersion has a total surface of greater than about 5000 m^{2/}/kg , at least about 5500 m^{2/}/kg, about 5500 m^{2/}/kg to about 12000 m^{2/}/kg, or about 7000 m^{2/}/kg to about 12,000 m^{2/}/kg. In certain embodiments, these values are determined using a commercially available particle size analyzer, such as Malvern Instrument Model 3000, Mastersizer 3000 using standard settings or another suitable device.

### Uses/Treatments

In certain embodiments, the nanoparticle ATQ is dosed as a sole active ingredient. Pharmaceutically acceptable compositions are provided which comprise the nanoparticle ATQ and at least one pharmaceutically acceptable dispersant based on the total ATQ dispersion, having a particle distribution of: wherein at least about 90% of the ATQ nanoparticles have a volume diameter below about 0.776 μm, as determined using dynamic light scattering (DLS), at least about 50% of the nanoparticles have a volume diameter below about 0.549 μm; at least about 10% of the nanoparticles have a volume diameter below about 0.393 μm; and (b) an optional pharmaceutically acceptable carrier or excipient. In certain embodiments, the ATQ nanoparticles have an average surface area in excess of about 5500 m²/kg.

Pharmaceutical formulations include those suitable for oral and parenteral (including subcutaneous, intradermal, intramuscular and intravenous) administration as well as administration by naso-gastric tube. Suitable formulations within the scope of the present invention include, for example, solid dosage forms such as tablets and liquid dosage forms, such as suspensions. The formulation may, where appropriate, be conveniently presented in discrete dosage units and may be prepared from the ATQ nanoparticles described herein using methods known in the art of pharmacy.

The pharmaceutically acceptable compositions provided herein may be a powder, powder for suspension, powder in capsule, suspension, capsule, tablet, or other suitable form. Advantageously, because the nanoparticle ATQ is more bioavailable that the commercially available liquid product on the market, it can be delivered in a lower dose, having a lower concentration.

In certain embodiments, tablets provided herein, permit tablet doses which are lower than the discontinued commercial product which typically involved a dose of three 250 mg tablets (750 mg/dose) having a size of 10 mm three times a day (2250 mg/day). In certain embodiments, a tablet containing nanoparticle ATQ as the sole active pharmaceutical ingredient (API) herein may be delivered in a daily dose of 500 mg/day to about 1500 mg/day, or about 750 mg/day to 1250 mg/day. Such tablets may each contain 50 mg, 100 mg, or 250 mg nanoparticle ATQ, or another suitable amount and may be delivered alone or as multiple tablets once, twice or multiple times per day.

A variety of inactive ingredients may be selected for inclusion in the tablet, including, e.g., filler, one or more disintegrant, one or more binder, one or more a buffering agent, one or more dispersants, one or more lubricant, one or more glidant, or blends of these components. Suitably, the tablets also include taste and/or mouth feel enhancers including, e.g., one or more of a sweetener, a flavorant, a gum, or blends of these components. Optionally, the tablet may also contain a moisture barrier coating. In general, such a coating is selected which provides no detectable modified release functions. The non-functional coating may be a polymer which may serve as a moisture barrier to preserve the integrity of the tablet during storage or to facilitate application of a color coating layer. Additionally or alternatively, a coating may be selected which provides a color coating layer or improve the “smoothness” or mouth feel of the tablet. Examples of suitable tablet components may include, without limitation, dispersants, hydroxypropyl cellulose, hydroxypropylmethylcellulose, magnesium stearate, microcrystalline cellulose, polyethylene glycol, povidone, sodium starch glycolate, colorants, flavorant.

In certain embodiments, an ATQ composition is a liquid suspension for oral administration. Examples of suitable suspending agents may include, e.g., water, buffered saline, methyl cellulose and xanthan gum, which may be combined with an aqueous suspension base containing water and an optional solvent. In one embodiment, the suspending agent is xanthan gum. The suspension base may include, e.g., flavorant, sweeteners, and among other excipients. In certain embodiments, a suspension medium comprises a viscosity enhancing material, e.g., a gum dispersion, which assists in ensuring that the drug particles remain suspended, do not settle and do not form agglomerates. Such a gum dispersion may be prepared separately. The gum is typically a colloidal dispersion which may be milled to a size desired to improve function or mouth feel. In certain embodiments, no gum dispersion is required. In certain embodiments, a liquid suspension includes water, benzyl alcohol, a sweetener, xanthan gum, an optional flavorant (e.g., citrus), and an optional dye or colorant. In certain embodiments, a liquid suspension includes water, benzyl alcohol, a sweetener, guar gum, an optional flavorant (e.g., citrus), and an optional dye or colorant. Other suitable excipients, at least one dispersant, surfactants, viscosity enhancing agents, or components will be apparent to one of skill in the art.

In certain embodiments, the ATQ composition has an oral dose of about 1 mg/kg to about 12 mg/kg. In certain embodiments, the nanoparticle ATQ composition is an oral liquid suspension comprising about 25 mg/mL to about 100 mg/mL ATQ.

The reduction of the ATQ into the nano size range also enables the use of the compositions provided herein in injectable dosage forms. While a variety of suspending agents may be selected, including purified water adjusted to physiologically compatible pH (e.g., about 6.8 to about 7.2). In certain embodiments, a buffered saline is selected. In certain embodiments, a preservative may be included. Such injectables may be delivered by any suitable route, including, intravenous, intramuscular, intraperitoneal, subcutaneous, or another selected route. Other suitable excipients may be selected.

In certain embodiments, the ATQ suspension for injectable administration comprises purified water and an optional buffering agent. In certain embodiments, the injectable composition is delivered at a dose of about 0.01 mg/kg body weight ATQ to about 10 mg/kg body weight ATQ. In certain embodiments, ATQ injectable suspension comprises about 10 mg/mL ATQ to about 75 mg/mL ATQ.

In certain embodiments, ATQ is combined with another active ingredient or co-administered with another active ingredient. The combination of ATQ and proguanil (also known as chloroguanide and chloroguanide) may conveniently be presented as a pharmaceutical formulation in unit dosage form. Proguanil has the chemical name N-(4-chlorophenyl)-N′-(1-methylethyl) imidodicarbonimidic diamide. It is an antiprotozoal agent having the structure:

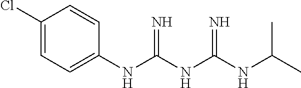 [SMILES: CC(C)NC(=N)NC(=N)Nc1ccc(Cl)cc1]

Proguanil also has the chemical name 1-(4-chlorophenyl)-5-isopropyl-biguanide. It is commercially available as a hydrochloride salt. Proguanil is an antiprotozoal drug. Other antiprotozoal drugs include cycloguanil, mefloquinine, quinine, amodiaquine, chloroquine, hydroxychloroquine, pamaquine, primaquine, pyrimethamine, artemisinin, artemether, artesunate, artenimol, artemotil, halofantrine, lumefantrine and pharmaceutically acceptable salts thereof. A convenient unit dose formulation contains the active ingredients in amounts of from 10 mg to 3 g each, e.g. 50 mg to 3 g each. Typical unit doses may contain for example 500 mg of ATQ and 200 mg of proguanil or 500 mg of ATQ and 500 mg of proguanil. However, given the improved bioavailability of the ATQ provided herein, the compositions provided herein may contain lower doses of ATQ e.g., about 250 mg to about 350 mg of ATQ. However, other doses may be selected. In certain embodiments, when used to treat malaria, the combination of proguanil: ATQ is in the ratio of 1:0.2 to 1:10. It should be understood that the dosages referred to above are calculated in terms of the drugs per se, not the salt forms.

In certain embodiments, ATQ may be combined with rifabutin, e.g., to treat Pneumocystis carinii infections. Rifabutin is a semisynthetic ansamycin antibiotic derived from rifamycin S. It has the chemical name 1′,4-didehydro-1-deoxy-1,4-dihydro-5′-(2-methylpropyl)-1-oxorifamycin XIV (Chemical Abstracts Service, 9th Collective Index) or (9S, 12E, 14S, 15R, 16S, MR, 18R, 19R, 20S, 21 S, 22E, 24Z)-6,16,18,20-tetrahydroxy-1′-isobutyl-14-methoxy-7,9,15,17,19,21,25-heptamethyl-spiro[9,4-(epoxypentadeca[1,11,13]trienimino)-2H-furo[2\3\:7,8]naphth[1,2-c midazole-2,4′-piperidine]-5_{I}10,26-(3H,9H)-trione-16-acetate. Its structure is represented by Formula (I).

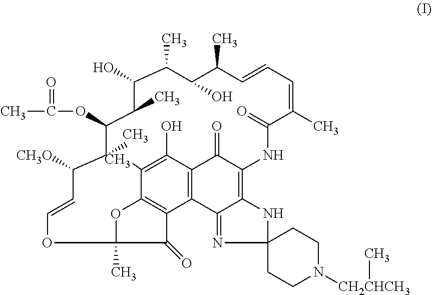

In general, a suitable effective dose for administration to man for treatment of Pneumocystis carinii infections is in the range of 5 mg to 1000 mg of ATQ per kilogram bodyweight per day and 5 mg to 1000 mg of rifabutin per kilogram bodyweight per day, for example from 10 to 500 mg/kg/day of ATQ and 20 to 500 mg/kg/day of rifabutin, particularly 20 to 100 mg/kg/day of ATQ and 20 to 400 mg/kg/day of rifabutin. A suitable effective dose for administration to man for prophylaxis of Pneumocystis carinii infections is in the range of from 100 to 10,000 mg per kilogram bodyweight per week of each of ATQ and rifabutin for example from 200 to 3000 mg/kg/week of each of ATQ and rifabutin. However, given the improved bioavailability of the ATQ provided herein, the compositions provided herein may contain doses of ATQ below 200 to 3000 mg/kg/week, e.g., about 100 mg to about 2000 mg of ATQ. It should be understood that the dosages referred to above are calculated in terms of the drugs per se, not the salt forms. The combination of ATQ and rifabutin may conveniently be presented as a pharmaceutical formulation in unit dosage form. A convenient unit dose formulation contains the active ingredients in amounts of from 10 mg to 3 g each, e.g. 50 mg to 3 g each. Typical unit doses may contain for example 100 mg of ATQ and 300 mg of rifabutin or 50 mg of ATQ and 150 mg of rifabutin. In certain embodiments, the doses of ATQ may be lowered in view of the improved bioavailability to a dose below 50 mg, e.g., to 15 mg to 45 mg.

In certain embodiments, the ATQ nanoparticle composition described herein may be used in treatment of Babesia, Human Immunodeficiency Virus (HIV) infection, and/or associated with AIDS. In such treatments, the ATQ nanoparticle composition may be the sole treatment, or it may be co-administered with azithromycin. There are also certain infections of companion animals such as dogs and cats, including parasitic and/or tick-born infections, which are treatable with ATQ or the combination of ATQ and azithromycin.

In certain embodiments, an ATQ nanoparticle composition may be delivered in an anti- malarial regimen, or other regimen alone or in combination with another active agent in human and for veterinary uses, including, but not limited to companion animals such as dogs and cats.

In certain embodiments, a method for treating malaria in a human is provided which comprises administering a therapeutically effective amount of a suspension comprising ATQ as described herein.

In certain embodiments, a method for treatment of a Babesia, HIV infection, and/or AIDS is provided which comprises co-administering a ATQ suspension as provided herein to a patient in need thereof. In certain embodiments, azithromycin is co-administered with the ATQ composition.

In certain embodiments, a method for treatment of parasitic and/or tick-borne infections of companion animals is provided which comprises administering a composition comprising ATQ as provided herein to an animal in need thereof. In certain embodiments, azithromycin is co-administered with the ATQ composition.

In certain embodiments, a method for treating Pneumocystic carinii infections is provided which comprises co-administering a composition comprising ATQ as described herein and rifabutin. In certain embodiments, the rifabutin is in the ATQ suspension. In other embodiments, the rifabutin is formulated separately from the ATQ suspension.

The following examples are illustrative of the ATQ nanosuspensions of the invention.

### EXAMPLES

### Example 1: ATQ Oral Dosage Forms

A. Preparation of ATQ Dispersion with Poloxamer 188

| Ingredient | Quantity |
|---|---|
| ATQ Dispersion | |
| ATQ | 250 g |
| Poloxamer 188 | 30 g |
| Purified Water | 500 g |

ATQ dispersion was prepared by first dissolving 30 g of poloxamer 188 in 500 g of purified water in a stainless steel jacketed container, followed by adding 250 g of ATQ to achieve a uniform dispersion with overhead stirrer. Separately, gum dispersion was prepared by combining 8.33 g of xanthan gum, 3.33 g of poloxamer 188, 16.7 g of benzyl alcohol, 3.0 g of saccharin sodium and 800 g of purified water.

The ATQ dispersion was milled using an immersion mill having a chamber with porous wall and the temperature was maintained at 22° C. to 28° C. A stir paddle was used to improve the recirculation during the milling process. About 70% v/v grinding media was loaded into the chamber. The grinding media used in this example was Zirconia Oxide, 0.3 mm. The mixture was recirculated continuously through the chamber with milling speed at 3000 rpm for 20 minutes to generate the milled ATQ particles dispersion.

B. Preparation of ATQ Oral Suspension

| Ingredient | Quantity | |
|---|---|---|
| Gum Dispersion | | |
| Xanthan gum | 8.33 | g |
| Benzyl Alcohol | 16.7 | g |
| Saccharin Sodium | 3.00 | g |
| Poloxamer 188 | 3.33 | g |
| Purified Water | 800 | g |
| ATQ Suspension | | |
| ATQ Dispersion | 400 | g |
| Gum Dispersion | 427 | g |
| Flavor Tutti Frutti | 0.17 | g |
| Purified Water | QS 871.8 | g |

400 g of milled ATQ dispersion prepared as described in Part A of this Example was mixed with 427 g of gum dispersion, followed by adding 0.17 g flavor tutti frutti. The final suspension (Test 1) was obtained by adjusting the total weight to 871.8 g with appropriate amount of purified water and mixed until uniform.

C. Preparation of ATQ Solid Dosage Forms

An ATQ dispersion can be prepared as described in Part A of this Example and freeze dried (lyophilized) to a dry powder, optionally blended with excipients (e.g., flow agents), and loaded into a capsule or packaging into a suitable pouch to be prepared at a later date as an oral suspension by a pharmacist or patient.

Alternatively, an ATQ dispersion can be prepared as described in Part A of this Example and freeze dried (lyophilized) to a dry powder, optionally blended with other excipients, and compressed into a tablet or loaded into a capsule.

### Example 2: Preparation of ATQ Suspension

| Ingredient | Quantity | |
|---|---|---|
| ATQ Dispersion | | |
| ATQ | 250 | g |
| Poloxamer 188 | 30 | g |
| Purified Water | 500 | g |
| Gum Dispersion | | |
| Xanthan gum | 8.33 | g |
| Benzyl Alcohol | 16.7 | g |
| Saccharin Sodium | 3.00 | g |
| Poloxamer 188 | 3.33 | g |
| Sodium Lauryl Sulfate | 1.67 | g |
| Purified Water | 800 | g |
| ATQ Suspension | | |
| ATQ Dispersion | 400 | g |
| Gum Dispersion | 427 | g |
| Flavor Tutti Frutti | 0.17 | g |
| Purified Water | QS 871.8 | g |

ATQ dispersion was prepared by first dissolving 30 g of poloxamer 188 in 500 g of purified water in a stainless steel jacketed container, followed by adding 250 g of ATQ to achieve a uniform dispersion with overhead stirrer. Separately, gum dispersion was prepared by combining 8.33 g of xanthan gum, 3.33 g of poloxamer 188, 1.67 g of sodium lauryl sulfate, 16.7 g of benzyl alcohol, 3.0 g of saccharin sodium and 800 g of purified water.

The ATQ dispersion was milled using an immersion mill having a chamber with porous wall and the temperature was maintained at 22° C. to 28° C. A stir paddle was used to improve the recirculation during the milling process. About 70% v/v grinding media was loaded into the chamber. The grinding media used in this example was Zirconia Oxide, 0.2 mm. The mixture was recirculated continuously through the chamber with milling speed at 4000 rpm for first 60 minutes and 3000 rpm for additional 60 minutes to generate the milled ATQ particles dispersion. 400 g of milled ATQ dispersion was mixed with 428 g of gum dispersion followed by adding 0.17 g of flavor tutti frutti. The final suspension (Test 2) was obtained by adjusting the total weight to 871.8 g with appropriate amount of purified water and mixed until uniform.

### Example 3: Preparation of ATQ Suspension

| Ingredient | Quantity | |
|---|---|---|
| ATQ Dispersion | | |
| ATQ | 250 | g |
| Poloxamer 188 | 30 | g |
| Purified Water | 500 | g |
| Gum Dispersion | | |
| Xanthan gum | 11.7 | g |
| Benzyl Alcohol | 16.7 | g |
| Saccharin Sodium | 3.00 | g |
| Poloxamer 188 | 3.33 | g |
| Purified Water | 800 | g |
| ATQ Suspension | | |
| ATQ Dispersion | 300 | g |
| Gum Dispersion | 320 | g |

ATQ dispersion was prepared by first dissolving 30 g of poloxamer 188 in 500 g of purified water in a stainless steel jacketed container, followed by adding 250 g of ATQ to achieve a uniform dispersion with overhead stirrer. Separately, gum dispersion was prepared by combining 11.7 g of xanthan gum, 3.33 g of poloxamer 188, 16.7 g of benzyl alcohol, 3.0 g of saccharin sodium and 800 g of purified water.

The ATQ dispersion was milled using an immersion mill having a chamber with porous wall and the temperature was maintained at 22° C. to 28° C. A stir paddle was used to improve the recirculation during the milling process. About 70% v/v grinding media was loaded into the chamber. The grinding media used in this example was Zirconia Oxide, 0.3 mm. The mixture was recirculated continuously through the chamber with milling speed at 1500 rpm for 10 minutes to generate the milled ATQ particles dispersion. 300 g of milled ATQ dispersion was mixed with 320 g of gum dispersion. The final suspension was mixed until uniform.

### Example 4: Pharmacokinetics of ATQ Nanosuspension

A 12-subject 3-way crossover pharmacokinetic study on healthy volunteers has been conducted with 2 prototype ATQ suspensions of different particle sizes versus a reference drug product (Mepron®). Chemical stability of the bio batches have been monitored and excellent chemical and physical stabilities have been obtained throughout the accelerated stability study. No apparent particle size change and degradation were observed. The formulation with smaller particle size has significant increases in the bioavailability as reflected in the area under curve from the plasma time profile when compared to that of the larger particle size as well as the reference product. The feasibility of the technology has been proven to be successful in reducing the particle size and increasing the bioavailability.

An open label, randomized, three-period, three-treatment, three-sequence, single dose cross-over pharmacokinetic study of two test formulations (Test 1 and Test 2) of ATQ Oral Suspension 750 mg/5 mL and reference ‘MEPRON’ (ATQ) Oral Suspension 750 mg/5 mL in healthy adult human subjects was conducted under fasting conditions.

Particle size distribution of the test and reference products using Malvern Mastersizer® 3000 under the conditions of fixed duration (10 s), stir rate (1500 rpm) and ultrasound (15 s) is shown below.

| Formulation | Dᵥ (10), μm | Dᵥ (50), μm | Dᵥ (90), μm | Surface Area |
|---|---|---|---|---|
| Test 1 | 0.423 | 1.81 | 5.61 | 5501 |
| Test 2 | 0.393 | 0.549 | 0.776 | 11240 |
| Reference | 0.428 | 2.12 | 20.3 | 4708 |

TABLE
Pharmacokinetic Results for the Test and Reference Products

| | Geometric Mean | | | | |
|---|---|---|---|---|---|
| Pharmacokinetic | T₁ | T₂ | R | Test/Reference Ratio | |
| Parameter | (Test 1) | (Test 2) | (Reference) | T₁/R (%) | T₂/R (%) |
| Cₘₐₓ (μg/mL) | 3.036 | 4.935 | 1.570 | 193.36 | 314.29 |
| AUCₜ (μg.hr/mL) | 172.320 | 266.518 | 118.449 | 145.48 | 225.01 |
| AUGᵢ (μg.hr/mL) | 198.877 | 317.438 | 148.811 | 133.64 | 213.32 |

### Example 5: Preparation of ATQ Oral Dosage Form

A. ATQ Dispersion

| Ingredient | Quantity | |
|---|---|---|
| ATQ Dispersion | | |
| ATQ | 250 | g |
| Tween 80 | 1.67 | g |
| Purified Water | 500 | g |

ATQ dispersion is prepared by first dissolving 1.67 g of Tween 80 in 500 g of purified water in a stainless steel jacketed container, followed by adding 250 g of ATQ to achieve a uniform dispersion with overhead stirrer. The ATQ dispersion is milled as described in Example 1.

B. ATQ Oral Suspension

The ATQ dispersion prepared as described in Part A is used to prepare an oral suspension as follows.

| Ingredient | Quantity | |
|---|---|---|
| Gum Dispersion | | |
| Xanthan gum | 4.17 | g |
| Benzyl Alcohol | 8.35 | g |
| Saccharin Sodium | 3.0 | g |
| Purified Water | 823 | g |
| ATQ Suspension | | |
| ATQ Dispersion | 400 | g |
| Gum Dispersion | 444 | g |
| Flavor Tutti Frutti | 0.2 | g |
| Purified Water | QS 904.4 | g |

Separately from the ATQ dispersion, a gum dispersion is prepared by combining 4.17 g of xanthan gum, 8.35 g of benzyl alcohol, 3.0 g of saccharin sodium and 823 g of purified water. 400 g of the milled ATQ dispersion of Part A is mixed with 444 g of gum dispersion, followed by adding 0.2 g flavor tutti frutti. The final volume of the suspension is adjusted to target quantity by adding purified water and mixed until uniform.

C. Preparation of ATQ Solid Dosage Forms

An ATQ dispersion can be prepared as described in Part A of this Example and freeze dried (lyophilized) to a dry powder, optionally blended with excipients (e.g., flow agents), and loaded into a capsule or packaging into a suitable pouch to be prepared at a later date as an oral suspension by a pharmacist or patient.

The ATQ dispersion prepared as described in Part A of this Example can be used for drug layering on pellets and tablets/minitablets. Alternatively, an ATQ dispersion can be prepared as described in Part A of this Example and freeze dried (lyophilized) to a dry powder, optionally blended with other excipients, and compressed into a tablet or loaded into a capsule.

### Example 6: Preparation of ATQ Oral Dosage Forms

A. ATQ Dispersion

| Ingredient | Quantity | |
|---|---|---|
| ATQ Dispersion | | |
| ATQ | 250 | g |
| Tween 80 | 1.67 | g |
| Purified Water | 500 | g |

ATQ dispersion is prepared by first dissolving 1.67 g of Tween 80 in 500 g of purified water in a stainless steel jacketed container, followed by adding 250 g of ATQ to achieve a uniform dispersion with overhead stirrer. The ATQ dispersion is milled according to Example 1.

B. ATQ Suspension

The ATQ dispersion of Part A is used to prepare a suspension as follows.

| Ingredient | Quantity | |
|---|---|---|
| Gum Dispersion | | |
| Guar gum | 4.17 | g |
| Benzyl Alcohol | 16.7 | g |
| Saccharin Sodium | 2.0 | g |
| Purified Water | 800 | g |
| ATQ Suspension | | |
| ATQ Dispersion | 400 | g |
| Gum Dispersion | 444 | g |
| Flavor Tutti Frutti | 0.2 | g |
| Purified Water | QS 904.4 | g |

Separately, gum dispersion is prepared by combining 4.17 g of guar gum, 16.7 g of benzyl alcohol, 2.0 g of saccharin sodium and 800 g of purified water.

400 g of milled ATQ dispersion of Part A is mixed with 444 g of gum dispersion, followed by adding 0.2 g flavor tutti frutti. The final volume of the suspension is adjusted to target quantity by adding purified water and mixed until uniform.

C. Preparation of ATQ Solid Dosage Forms

An ATQ dispersion can be prepared as described in Part A of this Example and freeze dried (lyophilized) to a dry powder, optionally blended with excipients (e.g., flow agents), and loaded into a capsule or packaging into a suitable pouch to be prepared at a later date as an oral suspension by a pharmacist or patient.

Alternatively, an ATQ dispersion can be prepared as described in Part A of this Example and freeze dried (lyophilized) to a dry powder, optionally blended with other excipients, and compressed into a tablet or loaded into a capsule.

### Example 7: Preparation of ATQ Oral Dosage Forms

A. ATQ Dispersion

| Ingredient | Quantity | |
|---|---|---|
| ATQ Dispersion | | |
| ATQ | 250 | g |
| TPGS | 16.7 | g |
| Purified Water | 500 | g |

ATQ dispersion is prepared by first dissolving 16.7 g of TPGS in 500 g of purified water in a stainless steel jacketed container, followed by adding 250 g of ATQ to achieve a uniform dispersion with overhead stirrer. The ATQ dispersion is milled according to Example 1.

B. ATQ Suspension

The ATQ dispersion of Part A is used to prepare a suspension as follows.

| Ingredient | Quantity | |
|---|---|---|
| Gum Dispersion | | |
| Xanthan gum | 13.3 | g |
| Benzyl Alcohol | 25.0 | g |
| Saccharin Sodium | 2.0 | g |
| Purified Water | 800 | g |
| ATQ Suspension | | |
| ATQ Dispersion | 445 | g |
| Gum Dispersion | 448 | g |
| Flavor Tutti Frutti | 0.17 | g |
| Purified Water | QS 975.8 | g |

Separately, gum dispersion is prepared by combining 13.3 g of xanthan gum, 25.0 g of benzyl alcohol, 2.0 g of saccharin sodium and 800 g of purified water. 445 g of milled ATQ dispersion is mixed with 448 g of gum dispersion, followed by adding 0.17 g flavor tutti frutti. The final volume of the suspension is adjusted to target quantity by adding purified water and mixed until uniform.

C. Preparation of ATQ Solid Dosage Forms

An ATQ dispersion can be prepared as described in Part A of this Example and freeze dried (lyophilized) to a dry powder, optionally blended with excipients (e.g., flow agents), and loaded into a capsule or packaging into a suitable pouch to be prepared at a later date as an oral suspension by a pharmacist or patient.

Alternatively, an ATQ dispersion can be prepared as described in Part A of this Example and freeze dried (lyophilized) to a dry powder, optionally blended with other excipients, and compressed into a tablet or loaded into a capsule.

### Example 8: Preparation of ATQ Injectable

| Ingredient | Quantity | |
|---|---|---|
| ATQ Dispersion | | |
| ATQ | 250 | g |
| Benzalkonium chloride | 16.7 | g |
| Sterile Water for injection/Saline | 500 | g |
| solution | | |
| ATQ Suspension | | |
| ATQ Dispersion | 445 | g |
| Phosphate Buffered Saline | QS 975.8 | g |

ATQ dispersion is prepared by first dissolving 16.7 g TPGS in 500 g of Saline solution or water for injection in a stainless steel jacketed container, followed by adding 250 g of ATQ to achieve a uniform dispersion with overhead stirrer.

The ATQ dispersion was milled using an immersion mill having a chamber with porous wall and the temperature was maintained at 22° C. to 28° C. A stir paddle was used to improve the recirculation during the milling process. About 70% v/v grinding media was loaded into the chamber. The grinding media used in this example was Zirconia Oxide, 0.3 mm. The mixture was recirculated continuously through the chamber with milling speed at 3000 rpm for 20 minutes to generate the milled ATQ particles dispersion.

B. Preparation of ATQ Injectable Suspension

The ATQ injectable preparation is prepared by utilizing the dispersion prepared as per Part A of the Example and subjecting to terminal sterilization (Autoclave), lyophilization (Class 100) and aseptic filling (Class 100). For the lyophilized powder at the time of administration, it is reconstituted using water for injection or saline solution and mixed until uniform.

### Example 9: Preparation of ATQ—Proguanil Composition (250 mg/100 mg/5 ml)

| Ingredient | Quantity | |
|---|---|---|
| ATQ Dispersion | | |
| ATQ | 250 | g |
| Poloxamer 188 | 30 | g |
| Purified Water | 600 | g |
| Gum Dispersion | | |
| Xanthan gum | 19.4 | g |
| Benzyl Alcohol | 38.7 | g |
| Saccharin Sodium | 5.9 | g |
| Poloxamer 188 | 3.0 | g |
| Purified Water | 2933 | g |
| ATQ/Proguanil Suspension | | |
| ATQ Dispersion | 500 | g |
| Proguanil HCl | 56.8 | g |
| Gum Dispersion | 2200 | g |
| Flavor Tutti Frutti | 0.56 | g |
| Purified Water | QS to 2840 | ml |

ATQ/Proguanil HCl dispersion is prepared by first dissolving 30 g of poloxamer 188 in 500 g of purified water in a stainless steel jacketed container, followed by adding 250 g of ATQ to achieve a uniform dispersion with overhead stirrer. Separately, gum dispersion is prepared by combining 19.4 g of xanthan gum, 38.7 g of benzyl alcohol, 5.9 g of saccharin sodium, 3.0 g of poloxamer 188 and 2933 g of purified water.

The ATQ is milled according to Example 2. 500 g of milled ATQ dispersion is mixed with 2200 g of gum dispersion, followed by adding 56.8 g of proguanil hydrochloride and 0.56 g flavor tutti frutti. The final volume of the suspension is adjusted to target quantity by adding purified water and mixed until uniform.

All patents, patent publications, and other publications listed in this specification, are incorporated herein by reference. U.S. Provisional Application No. 62/747,504, filed Oct. 18, 2018, is incorporated herein by reference in its entirety. While the invention has been described with reference to a particularly preferred embodiment, it will be appreciated that modifications can be made without departing from the spirit of the invention. Such modifications are intended to fall within the scope of the appended claims.

1. A nanoparticle atovaquone composition suitable for administration comprising:
(a) an atovaquone dispersion comprising atovaquone nanoparticles and at least one nanoparticle pharmaceutically acceptable dispersant based on the total atovaquone dispersion, having a particle distribution of:
wherein at least about 90% of the atovaquone nanoparticles have a volume diameter below about 0.776 μm, as determined using dynamic light scattering (DLS),
at least about 50% of the nanoparticles have a volume diameter below about 0.549 μm;
at least about 10% of the nanoparticles have a volume diameter below about 0.393 μm; and
(b) an optional pharmaceutically acceptable carrier or excipient.

2. The nanoparticle atovaquone composition according to claim 1, wherein atovaquone nanoparticles have an average surface area in excess of about 5500 m²/kg.

3. The nanoparticle atovaquone composition according to claim 1, which provides a pharmacokinetic profile for atovaquone having a maximum plasma atovaquone concentration (Cmax) value of 3 μg/mL to 6 μg/mL.

4. The nanoparticle atovaquone composition according to claim 1, which provides a pharmacokinetic profile for atovaquone having an area under the curve of (AUC) inf of 317.438 μg·hr/mL under fasted conditions.

5. The nanoparticle atovaquone composition according to claim 1, wherein the at least one nanoparticle dispersant is a surfactant, viscosity enhancing material or an ionic species.

6. The nanoparticle atovaquone composition according to claim 5, wherein the surfactant is a poloxamer.

7. The nanoparticle atovaquone composition according to claim 6, wherein the poloxamer is selected from poloxamer 188, poloxamer 238, or poloxamer 407.

8. The nanoparticle atovaquone composition according to claim 1, wherein the composition further comprises a second dispersant, surfactant, or a viscosity enhancing material.

9. The nanoparticle atovaquone composition according to claim 1, wherein the composition is a solid or a liquid.

10. The nanoparticle atovaquone composition according to claim 1, which is formulated for oral administration.

11. The nanoparticle atovaquone composition according to claim 1, wherein the atovaquone has an oral dose of about 1 mg/kg to about 12 mg/kg.

12. The nanoparticle atovaquone composition according to claim 1, wherein the formulation is for injectable administration and further comprises purified water and an optional buffering agent.

13. The nanoparticle atovaquone composition according to claim 12, wherein the injectable composition is delivered at a dose of about 0.01 mg/kg body weight atovaquone to about 10 mg/kg body weight atovaquone.

14. The nanoparticle atovaquone composition according to claim 12, wherein the atovaquone is in an oral liquid comprising of about 25 mg/mL to about 100 mg/mL.

15. The nanoparticle atovaquone composition according to claim 12, wherein the atovaquone is in an injectable liquid comprising of about 10 mg/mL to about 75 mg/mL.

16. The nanoparticle atovaquone composition according to claim 1, wherein the atovaquone is in an injectable liquid comprising of about 10 mg/mL to about 75 mg/mL.

17. The nanoparticle atovaquone composition according to claim 1, which is formulated as a powder for suspension, tablet or capsule.

18. A nanoparticle atovaquone powder comprising:
(a) atovaquone nanoparticles having a particle distribution of:
at least about 90% of the atovaquone nanoparticles have a volume diameter below about 0.776 μm, as determined using dynamic light scattering (DLS),
at least about 50% of the nanoparticles have a volume diameter below about 0.549 μm;
at least about 10% of the nanoparticles have a volume diameter below about 0.393 μm, and
(b) at least one dispersant.

19. A suspension comprising the powder according to claim 18.

20. A capsule comprising the powder according to claim 18.

21. A pharmaceutically acceptable composition comprising a combination of nanoparticle atovaquone and proguanil:
(a) an atovaquone dispersion comprising atovaquone nanoparticles and at least one nanoparticulate pharmaceutically acceptable surfactant, wherein based on the total atovaquone dispersion, having a particle distribution of:
wherein at least about 90% of the atovaquone nanoparticles have a volume diameter below about 0.776 μm, as determined using dynamic light scattering (DLS),
at least about 50% of the nanoparticles have a volume diameter below about 0.549 μm; and
at least about 10% of the nanoparticles have a volume diameter below about 0.393 μm.
(b) proguanil.

22. The pharmaceutically acceptable composition according to claim 21, wherein the atovaquone has a dose is about 1 mg/kg to about 12 mg/kg.

23. The pharmaceutically acceptable composition according to claim 21, wherein the proguanil is a nanoparticle.

24. (canceled)

25. A method for treating malaria in a human comprising administering a therapeutically effective amount of a composition according to claim 1.

26. A method for treatment of a Babesia, HIV infection, and/or AIDS which comprises administering a composition comprising atovaquone according to claim 1, optionally in a combination therapy.

27. The method according to claim 26, wherein the combination therapy comprises co-administering azithromycin.

28. A method for treatment of parasitic and/or tick-borne infections of companion animals which comprises administering a composition according to claim 1 to an animal in need thereof.

29. The method according to claim 28, wherein azithromycin is co-administered with the atovaquone composition.

30. A method for treating Pneumocystic carinii infections which comprises co-administering a composition according to claim 1 and rifabutin.

31. The method according to claim 30, wherein the rifabutin is in the atovaquone composition.

32. The method according to claim 31, wherein the rifabutin is formulated separately from the atovaquone composition.
